(19) United States
(12) Patent Application Publication
(10) Pub. No.: US 2010/0144749 A1
(43) Pub. Date: Jun. 10, 2010

(54) TREATMENT OF CANCERS WITH ACQUIRED RESISTANCE TO KIT INHIBITORS

(76) Inventors: Scott Wilhelm, Morristown, NJ (US); Gedrich Richard, Louisville, CO (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/093,515

(22) PCT Filed: Nov. 14, 2006
(86) PCT No.: PCT/US06/44237

§ 371 Date: Nov. 13, 2008

Related U.S. Application Data
(60) Provisional application No. 60/735,852, filed on Nov. 14, 2005.
(60) Provisional application No. 60/787,692, filed on Mar. 31, 2006.

(51) Int. Cl. A61K 31/44; A61K 31/506; A61P 35/00; A61P 35/02

(52) U.S. Cl. 514/252.18; 514/346

(57) ABSTRACT

The present invention provides compositions and methods for treating cancers which have acquired resistance to a KIT inhibitor by administering effective amounts of sorafenib.

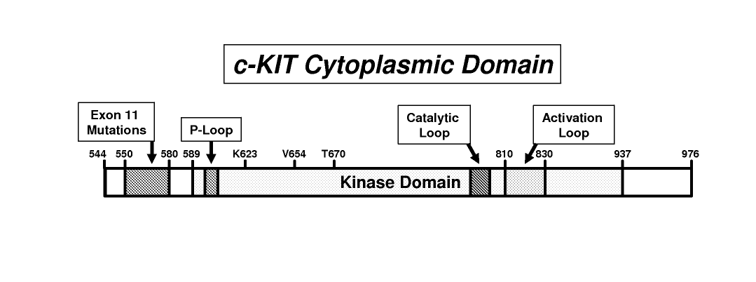

### CROSS-REFERENCE TO RELATED APPLICATIONS

This application claims the benefit of earlier-filed U.S. Provisional Application Ser. Nos. 60/735,852, filed Nov. 14, 2005 and 60/787,692, filed Mar. 31, 2006 which are incorporated herein by reference in its entirety.

### DESCRIPTION OF THE INVENTION

Cancer is a class of diseases characterized by two heritable properties: (1) uncontrolled cell division and (2) the ability of these cells to invade other tissues, either by direct growth into adjacent tissue (invasion) or by migration of cells to distant sites (metastasis). The hyper-proliferative properties initially give rise to a tumor or neoplasm. A tumor is considered a cancer when its cells acquire the ability to invade surrounding tissues, e.g., by breaking loose and entering the blood or lymph systems, or by forming secondary tumors at other sites in the body. The unregulated growth is caused by damaged DNA, resulting in mutations to vital genes that control cell division, the cell cycle, among other functions. One or more of these mutations, which can be inherited or acquired, can lead to uncontrolled cell division and cancer.

Cancers can be classified according to the tissue and cell type from which they arise. Cancers developing from epithelial cells are called carcinomas, and those from connective and muscle cells are called sarcomas. Additional cancers include those arising from hematopoietic cells (e.g., leukemia) and cancers of the nervous system.

In general, cancers appear to arise during a process in which an initial population of abnormal cells evolve into more aberrant cells through successive cycles of mutation and selection. More than 100 different genes have been identified which, when mutant, result in cancer. These so-called cancer-critical genes fall into two broad classes: oncogenes and tumor suppressor genes. Many cancer-critical genes play a role in the regulation of cell divisions, a highly complicated process involving multiple and parallel pathways. These include growth factors, cytokines, hormones, etc.

Cancer can cause many different symptoms, depending on the site and character of the malignancy and whether there is metastasis. A definitive diagnosis usually requires the microscopic examination of tissue obtained by biopsy. Once diagnosed, cancer is usually treated with surgery, chemotherapy and/or radiation.

If untreated, most cancers eventually cause death. Cancer is one of the leading causes of death in developed countries. It is estimated by the National Cancer Institute that approximately 9.8 million Americans were alive in January 2001 with a history of cancer. About 1,372,910 new cases of cancer were expected to be diagnosed in 2005, alone. In 2005, almost 600,000 Americans died of cancer, about 1 out of every 4 deaths. Many forms of cancer are associated with environmental factors, which may be avoidable. Smoking tobacco leads to more cancers than any other environmental factor.

Kinase inhibitors are being used successfully to treat cancers (e.g., Drevs et al., Current Drug Targets, 2003, 4, 113-121). However, some patients acquire a resistance to the drug's activity. In one embodiment, the present invention provides methods of treating a cancer in a subject in need thereof, comprising administering an effective amount of sorafenib to a subject having a cancer, wherein said cancer has acquired resistance to a KIT tyrosine kinase inhibitor. A tyrosine kinase inhibitor is a drug (i.e., a chemical compound) that blocks or reduces its kinase activity. Generally, a “tyrosine kinase activity” refers to the ability of the tyrosine kinase to auto-phosphorylate itself or trans-phosphorylate receptor subunits (or other substrates) by catalyzing the transfer of a phosphate from ATP (or another phosphate donor) to a tyrosine residue.

There are a number of well-documented instances where cancers have acquired resistance to a kinase inhibitor which previously had successfully been used to treat the cancer. The term “acquired resistance” indicates that the cancer becomes resistant and/or substantially less response to the effects of the drug after being exposed to it for a certain period of time. For example, gastrointestinal stromal tumors (GIST), a mesenchymal tumor of the intestinal tract, and chronic myelogenous leukemia (CML) are treated with imatinib (STI571 or Gleevac), a tyrosine kinase inhibitor that inhibits the kinase activity of BCR-ABL, ABL, KIT, and PDGFR. It has shown been shown that, while patients may benefit from the treatment initially, many patients subsequently develop resistance to the agent. In some cases, this acquired resistance has been shown to result from a secondary mutation in the gene associated with the cancer. For example, many GIST patients have an activating mutation in either the KIT or PDGFRA gene. A study of GIST patients with acquired resistance to imatinib showed secondary mutations in the KIT kinase domain. See, e.g., Antonescu et al, Clin. Cancer Res., 11(11):41824190, 2005 and Heinrich et al., J. Clin. Oncology, 24(29), 4764-4774, 2006 A second site mutation in BCR-ABL is the predominant mechanism of imiatinib resistance in CML. See, e.g., Gorre et al., Science, 293:876-880, 2001. Acquired resistance has also been observed with other cancer drugs, including patients treated with EGFR-kinase inhibitors, such as gefitinib (Iressa) or erlotinib (Tarceva). See, e.g., Kobayshi et al., N. Engl. J. Med., 352:786-792, 2005. Pao et al. (PLoS Med., 2, e73, 2005) observed that patients with progressing lung tumors contained, in addition to a primary drug-sensitive mutation in EGFR, a secondary mutation in the kinase domain which led to drug-resistance.

Examples of KIT inhibitors to which drug resistance can be acquired includes, but is not limited to, e.g., imatinib mesylate, and derivatives and salts thereof; PP1 (4-Amino-5-(4-methylphenyl)-7-(t-butyl)pyrazolo[3,4-d]pyrimidine); MLN518 (CT53518); PD180970; 5U112481 SU5416; SU5414; SU6597; SU6663; SU6561. See, also, Krystal et al., Cancer Res., 2001, 61:3660-3668.

Resistance mutations often occur in the kinase catalytic domain interfering or weakening the interaction with its inhibitor. Resistance secondary mutations for KIT have been reported. These secondary mutations often occur in the “gatekeeper” residue, the amino acid residue that “guards” the ATP-binding pocket and which also can comprise the site which interacts with the inhibitor. See, e.g., Noble et al., Science, 303: 1800-1805, 2004.

While not being bound to any mechanism, examples of mutations in the KIT gene which are associated with resistance or acquired resistance include, but are not limited to, e.g., mutations in Exons 13, 14, and or 17; mutations at residues 654, 670, 716, 816, 820, 822, and 823, residues about 650-654, residues about 670-674, residues about 816-824, in the A-loop (activation), such as V654A (Exon 13), T670I (Exon 14), T670E, D716N, S709F (Exon 14), D816G, D816E (Exon 17), C809G, D816H, D816V, D820A, D820E, D820Y, D820G N822K, Y823D (Exon 17), and/or deletions and other amino acid substitutions at such positions, or adjacent positions. Generally, any cancer having a primary and/or secondary KIT gene mutation associated with resistance or acquired resistance to a KIT inhibitor can be treated with a compound in accordance with the present invention.

As shown in the examples, mutations can decrease the affinity of a kinase inhibitor, such as imatinib, for the c-KIT protein, thereby decreasing the therapeutic efficacy of the drug. In contrast, where the drug potency for imatinib decreased from about 15- to over 2,000-fold, sorafenib potency was affected far less. Table 2 shows specific examples where its binding affinity decreased in some cases, but not all, and generally no more than about 15-fold. Any disorder in which the affected tissue (e.g., cancer) becomes resistant or less responsive to a KIT inhibitor can be treated with sorafenib or derivatives thereof.

KIT (also known as c-kit, mast cell growth factor receptor, or stem cell growth factor receptor) is the human homology of the provirus of the Hardy-Zuckerman 4 feline sarcoma virus. KIT encodes a transmembrane tyrosine kinase receptor which is expressed in a number of tissues, and is required for normal hematopoiesis, melanogenesis, and gametogenesis. The gene itself, is mapped to 4q11-q12, includes 21 exons, and is alternatively spliced. See, e.g., Vandenbark et al., Oncogene, 7:1259-1266, 1992.

Over-expression and/or gain-of-function mutations in KIT can result in ligand-independent tyrosine kinase activity, autophosphorylation of KIT, uncontrolled cell proliferation, and stimulation of downstream signaling pathways. For example, KIT was overexpressed in both malignant and benign gastrointestinal stromal tumors (GIST) tumors. See, e.g., Koon et al., Gut, 2004, 53:235-240. KIT is also expressed in acute myeloid leukemia, mast cell tumors, SCLC, germ cell tumors, breast cancer, and neuroblastoma.

Activating mutations in the KIT gene are associated with many types of GIST, the most common mesenchymal neoplasm in the human digestive tract. For example, Hirota et al., Science, 279:577-580, 1998, showed that of 49 mesenchymal tumors, 94% of them expressed an activated KIT. GISTs include a spectrum of tumors, including both benign and malignant types, and which occur at all levels of the gastrointestinal tract (e.g., stomach, small intestine, large intestine, rectum, etc.)

Cancers which are initially sensitive to a KIT inhibitor, but which have acquired resistance to it, can be treated in accordance with the present invention. Cancers having mutations in Exon 11 (from amino acid positions 550-582; see, e.g., Table 1) of the KIT gene are of particular relevance, and more preferably within codons 550-560. This region can also be referred to as the juxtamembrane domain. Specific examples include, but are not limited to: 1) deletion of amino acid residues 557-558; 2) deletion of amino acid residues 551-555; 3) deletion of amino acid residues 550-558; 4) deletion of amino acid residues 559-560; 5) deletion of amino acid residues 557-561; 6) deletion of amino acid residues 554-558; 7) deletion of amino acid residues 552-557; 8) mutations at residue 559, including V559D, V559A, or V559G; 9) mutations at residue 560, including V560D, V560E, or V560G; 10) W557S, alone, or in combination with a deletion of amino acids 552-556; 11) mutations at amino acid residue 557, including W557R; 12) mutations at amino acid residue 576, including L576P; 13) InsQL576-577. These mutations can be alone, or combined with other mutations, including with any of the specifically mentioned mutations. See, also, Lasota et al., Am. J. Path., 154:53-60, 1999.

Drug resistant cancers associated with other KIT mutations can be treated as well, especially those which are sensitive to KIT inhibitors. These include, e.g., systemic mastocytosis, e.g., having a F522C mutation (Akin et al., Blood, 2004, 193:3222-3225) and K509I (Zhang et al., 2005, Leuk. Res., Sept. 21); testicular seminomas, e.g., having imatinib mesylate sensitive mutations at amino acid residues 822 and 823, such as N822K and Y823D (e.g., Kemmer et al., Am. J. Pathol., 2004, 164:305-313, 2004).

Analysis of the gene mutations associated with cancer (e.g., GIST) having KIT mutation can be routinely determined. For example, PCR can be utilized to amplify specific regions using the published sequences of the human KIT gene. See, e.g., Andre et al., Genomics, 1997, 39:216-226. For amplification of Exon 11, see, e.g., Lasota et al., Am. J. Path., 154:53-60, 1999.

Nonetheless, the present invention relates to using sorafenib to treat a cancer, such as those mentioned above, which have acquired resistance to a KIT inhibitor, irrespective of the molecular mechanism responsible for it.

The present invention provides methods of treating cancers comprising, e.g., comprising administering to a subject in need thereof an effective amount of sorafenib, wherein the cancer is treated.

Examples of cancers that can be treated with imatinib, include, but not limited to: Accelerated Phase Chronic Myelogenous Leukemia; Acute Erythroid Leukemia; Acute Lymphoblastic Leukemia; Acute Lymphoblastic Leukemia in Remission; Acute Lymphocytic Leukemia; Acute Monoblastic and Acute; Monocytic Leukemia; Acute Myelogenous Leukemia; Acute Myeloid Leukemia; Adenocarcinoma of the Prostate; Adenoid Cystic Carcinoma of the Head and Neck; Advanced Gastrointestinal Stromal Tumor; Agnogenic Myeloid; Metaplasia; Anaplastic Oligodendroglioma; Astrocytoma; B-Cell Adult Acute Lymphoblastic Leukemia; Blastic Phase Chronic Myelogenous Leukemia; Bone Metastases; Brain Tumor; Breast Cancer; Cancer; Central Nervous System Cancer; Childhood Acute Lymphoblastic Leukemia; Childhood Acute Lymphoblastic Leukemia in Remission; Childhood Central Nervous System Germ Cell Tumor; Childhood Chronic Myelogenous Leukemia; Childhood Soft Tissue Sarcoma; Chordoma; Chronic Eosinophilic Leukemia (CEL); Chronic Idiopathic Myelofibrosis; Chronic Myelogenous Leukemia; Chronic Myeloid Leukemia; Chronic Myelomonocytic Leukemia; Chronic Phase Chronic Myelogenous Leukemia; Colon Cancer; Colorectal Cancer; Dermatofibrosarcoma; Dermatofibrosarcoma Protuberans (DFSP); Desmoid Tumor; Eosinophilia; Epidemic Kaposi's Sarcoma; Essential Thrombocythemia; Ewing's Family of Tumors; Extensive Stage Small Cell Lung Cancer; Fallopian Tube Cancer; Familiar Hypereosinophilia; Fibrosarcoma; Gastric Adenocarcinoma; Gastrointestinal Neoplasm; Gastrointestinal Stromal Tumor; Glioblastoma; Glioma; Gliosarcoma; Grade I Meningioma; Grade II Meningioma; Grade III Meningioma; Hematopoietic and Lymphoid Cancer; High-Grade Childhood Cerebral Astrocytoma; Hypereosinophilic Syndrome; Idiopathic Pulmonary Fibrosis; L1 Adult Acute Lymphoblastic Leukemia; L2 Adult Acute Lymphoblastic Leukemia; Leukemia, Lymphocytic, Acute L2; Leukemia, Myeloid, Chronic; Leukemia, Myeloid, Chronic Phase; Liver Dysfunction and Neoplasm; Lung Disease; Lymphoid Blastic Phase of Chronic Myeloid Leukemia; Male Breast Cancer; Malignant Fibrous Histiocytoma; Mastocytosis; Meningeal Hemangiopericytoma; Meningioma; Meningioma; Meningioma; Metastatic Cancer; Metastatic Solid Tumors; Myelofibrosis; Myeloid Leukemia, Chronic; Myeloid Leukemia, Chronic Accelerated-Phase; Myeloid Leukemia, Chronic, Chronic-Phase; Myeloid Metaplasia; Myeloproliferative Disorder (MPD) with Eosinophilia; Neuroblastoma; Non-T, Non-B Childhood Acute Lymphoblastic Leukemia; Oligodendroglioma; Osteosarcoma; Ovarian Germ Cell Tumor; Ovarian Low Malignant Potential Tumor; Ovarian Neoplasms; Pancreatic Cancer; Pelvic Neoplasms; Peritoneal Cavity Cancer; Peritoneal Neoplasms; Philadelphia Chromosome Positive Chronic Myelogenous Leukemia; Philadelphia Positive Acute Lymphoblastic Leukemia; Philadelphia Positive Chronic Myeloid Leukemia in Myeloid Blast Crisis; Polycythemia Vera; Pulmonary Fibrosis; Recurrent Adult Brain Tumor; Recurrent Adult Soft Tissue Sarcoma; Recurrent Breast Cancer; Recurrent Colon Cancer; Recurrent Esophageal Cancer; Recurrent Gastric Cancer; Recurrent Glioblastoma Multiforme (GBM); Recurrent Kaposi's Sarcoma; Recurrent Melanoma; Recurrent Merkel Cell Carcinoma; Recurrent Ovarian Epithelial Cancer; Recurrent Pancreatic Cancer; Recurrent Prostate Cancer; Recurrent Rectal Cancer; Recurrent Salivary Gland Cancer; Recurrent Small Cell Lung Cancer; Recurrent Tumors of the Ewing's Family; Recurrent Uterine Sarcoma; Relapsing Chronic Myelogenous Leukemia; Rheumatoid Arthritis; Salivary Gland Adenoid Cystic Carcinoma; Sarcoma; Small Cell Lung Cancer; Stage II Melanoma; Stage II Merkel Cell Carcinoma; Stage III Adult Soft Tissue Sarcoma; Stage III Esophageal Cancer; Stage III Merkel Cell Carcinoma; Stage III Ovarian Epithelial Cancer; Stage III Pancreatic Cancer; Stage III Salivary Gland Cancer; Stage IIIB Breast Cancer; Stage IIIC Breast Cancer; Stage IV Adult Soft Tissue Sarcoma; Stage IV Breast Cancer; Stage IV Colon Cancer; Stage IV Esophageal Cancer; Stage IV Gastric Cancer; Stage IV Melanoma; Stage IV Ovarian Epithelial Cancer; Stage IV Prostate Cancer; Stage IV Rectal Cancer; Stage IV Salivary Gland Cancer; Stage IVA Pancreatic Cancer; Stage IVB Pancreatic Cancer; Systemic Mastocytosis; T-Cell Childhood Acute Lymphoblastic Leukemia; Testicular Cancer; Thyroid Cancer; Unresectable or Metastatic Malignant Gastrointestinal Stromal Tumor (GIST); Unspecified Adult Solid Tumor; Untreated Childhood Brain Stem Glioma; Uterine Carcinosarcoma, and Uterine Sarcoma.

The phrase “effective amount” indicates the amount of sorafenib which is effective to treat any symptom or aspect of the cancer. Effective amounts can be determined routinely. Further guidance on dosages and administration regimens is provided below.

The term “treating” is used conventionally, e.g., the management or care of a subject for the purpose of combating, alleviating, reducing, relieving, improving, etc., one or more of the symptoms associated with a cancer, including all cancers mentioned herein and in Table 1. Administering effective amounts of sorafenib can treat one or more aspects of the cancer disease, including, but not limited to, causing tumor regression; causing cell death; causing apoptosis; causing necrosis; inhibiting cell proliferation; inhibiting tumor growth; inhibiting tumor metastasis; inhibiting tumor migration; inhibiting tumor invasion; reducing disease progression; stabilizing the disease; reducing or inhibiting angiogenesis; prolonging patient survival; enhancing patient's quality of life; reducing adverse symptoms associated with cancer; and reducing the frequency, severity, intensity, and/or duration of any of the aforementioned aspects.

Any cancer can be treated in accordance of the present invention, irrespective of the type or cause of the cancer, and irrespective of the genetic lesions associated with. Examples of cancers which can be treated include, but are not limited to, GIST, acute myeloid leukemia, mast cell tumors, SCLC, germ cell tumors, breast cancer, neuroblastoma, sinonasal lymphoma, etc.

Cancers which can be treated include, e.g., cancers which are primary; which arise from a primary tumor at a secondary metastatic site; which have been treated by surgery (e.g., entirely removed, surgical resection, etc); which have been treated by chemotherapy, radiation, radio frequency ablation, and/or any other adjunct to drug therapy. Any subject can be in accordance with the present invention, including, e.g., mammals, such as mice, rats, dogs, cats, non-human primates, monkeys, and humans.

The ability of sorafenib to treat a cancer with acquired resistance to a KIT inhibitor can be routinely determined. For example, the IL-3-dependent murine hematopoietic cell line, Ba/F3, can be cultured independently of IL-3 when transfected with constitutively active KIT (e.g., having a deletion of amino acid residues 557-558). See, e.g., Tsujimura et al., Blood, 1999, 93:1319-1329. In the presence of a KIT inhibitor, such as imatinib, cells expressing the constitutively active KIT polypeptide undergo cell death as a result of KIT inhibition. The presence of a second mutation that confers resistance to the KIT inhibitor rescues the cells. Cells expressing the double-mutation (activating; KIT resistance) are cultured in the presence of sorafenib. Those cells which die are sensitive to sorafenib, indicating its usefulness in treating patients who have acquired resistance to the KIT inhibitor.

Specific examples of cancers which can be treated in accordance with the present invention include cancers have a deletion of residues 557-558, and which have at least one of the following mutations: V654A, T670I, D820Y, N822K, and Y823D.

The present invention also provides methods of determining whether to treat a subject having cancer with sorafenib, comprising determining the presence of a mutation in a KIT gene, wherein said mutation is an activating and/or KIT-inhibitor resistance mutation, and administering sorafenib to a subject having one or more pre-determined mutations. Activating and KIT-inhibitor resistance mutations have been described above. A subject who is resistant to a KIT inhibitor can be screened for the presence of an activating and/or resistance mutation (such as those listed above), a subject having the mutation(s) can be treated with sorafenib.

The term “sorafenib” as used herein refers to the tosylate salt of the compound N-[4-chloro-3-(trifluoromethyl)phenyl]-N′-{4-[2-carbamoyl-1-oxo-(4-pyridyloxy)]phenyl} urea of the formula I below including all polymorphs, hydrates, solvates or combinations thereof.

The compound N-[4-chloro-3-(trifluoromethyl)phenyl]-N′-{4-[2-carbamoyl-1-oxo-(4-pyridyloxy)]phenyl} urea of formula I below and all polymorphs, hydrates, solvates or combinations thereof are also suitable for use in this invention.

In addition, pharmaceutically acceptable salts of the compound of formula I, other than sorafenib, are also suitable for use in this invention, as are their polymorphs, hydrates, solvates or combinations thereof. Suitable pharmaceutically acceptable salts are well known to those skilled in the art and include salts of inorganic and organic acids, such as hydrochloric acid, hydrobromic acid, sulfuric acid, phosphoric acid, methanesulphonic acid, trifluoromethanesulfonic acid, benzenesulfonic acid, 1-naphthalenesulfonic acid, 2-naphthalenesulfonic acid, acetic acid, trifluoroacetic acid, malic acid, tartaric acid, citric acid, lactic acid, oxalic acid, succinic acid, fumaric acid, maleic acid, benzoic acid, salicylic acid, phenylacetic acid, and mandelic acid. In addition, pharmaceutically acceptable salts include salts of inorganic bases, such as salts containing alkaline cations (e.g., Li⁺ Na⁺ or K⁺), alkaline earth cations (e.g., Mg⁺², Ca⁺² or Ba⁺²), the ammonium cation, as well as acid salts of organic bases, including aliphatic and aromatic substituted ammonium, and quaternary ammonium cations, such as those arising from protonation or peralkylation of triethylamine, N,N-diethylamine, N,N-dicyclohexylamine, lysine, pyridine, N,N-dimethylaminopyridine (DMAP), 1,4-diazabiclo[2.2.2]octane (DABCO), 1,5-diazabicyclo[4.3.0]non-5-ene (DBN) and 1,8-diazabicyclo[5.4.0]undec-7-ene (DBU).

Solvates for the purposes of the invention are those forms of the compound where solvent molecules form a complex in the solid state and include, but are not limited to for example ethanol and methanol. Hydrates are a specific form of solvates, where the solvent molecule is water.

Formula I is as follows:

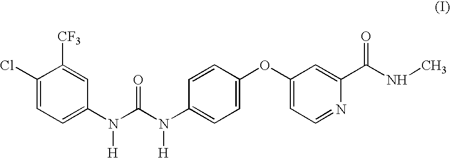

The compound N-[4-chloro-3-(trifluoromethyl)phenyl]-N′-{4-[2-carbamoyl-1-oxo-(4-pyridyloxy)]phenyl} urea can be prepared by the following multistep procedure:

### Step 1: Preparation of 4-chloro-2-pyridinecarboxamide

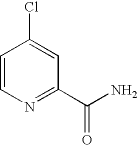

To a stirred mixture of methyl 4-chloro-2-pyridinecarboxylate hydrochloride (1.0 g, 4.81 mmol) dissolved in conc. aqueous ammonia (32 mL) is added ammonium chloride (96.2 mg, 1.8 mmol, 0.37 equiv.), and the heterogeneous reaction mixture is stirred at ambient temperature for 16 h. The reaction mixture is poured into EtOAc (500 mL) and water (300 mL). The organic layer is washed with water (2×300 mL) and a saturated NaCl solution (1×300 mL), dried (MgSO₄), concentrated in vacuo to give 4-chloro-2-pyridinecarboxamide as a beige solid (604.3 mg, 80.3%): TLC (50% EtOAc/hexane) R_{f} 0.20; ¹H-NMR (DMSO-d₆) δ 8.61 (d, J=5.4 Hz, 1H), 8.20 (broad s, 1H), 8.02 (d, J=1.8 Hz, 1H), 7.81 (broad s, 1H), 7.76 to 7.73 (m, 1H).

### Step 2: Preparation of 4-(4-aminophenoxy)-2-pyridinecarboxamide

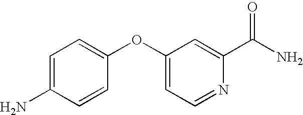

To 4-aminophenol (418 mg, 3.83 mmol) in anh DMF (7.7 mL) is added potassium tert-butoxide (447 mg, 3.98 mmol, 1.04 equiv.) in one portion. The reaction mixture is stirred at room temperature for 2 h, and a solution of 4-chloro-2-pyridinecarboxamide (600 mg, 3.83 mmol, 1.0 equiv.) in anh DMF (4 mL) is then added. The reaction mixture is stirred at 80° C. for 3 days and poured into a mixture of EtOAc and a saturated NaCl solution. The organic layer is sequentially washed with a saturated NH₄Cl solution then a saturated NaCl solution, dried (MgSO₄), and concentrated under reduced pressure. The crude product is purified using MPLC chromatography (Biotage®; gradient from 100% EtOAc to be followed by 10% MeOH/50% EtOAc/40% hexane) to give the 4-chloro-5-trifluoromethylaniline as a brown solid (510 mg, 58%). ¹H-NMR (DMSO-d₆) δ 8.43 (d, J=5.7 Hz, 1H), 8.07 (br s, 1H), 7.66 (br s, 1H), 7.31 (d, J=2.7 Hz, 1H), 7.07 (dd, J=5.7 Hz, 2.7 Hz, 1H), 6.85 (d, J=9.0 Hz, 2H), 6.62 (d, J=8.7 Hz, 2H), 5.17 (broad s, 2H); HPLC EI-MS m/z 230 ((M+H)⁺.

### Step 3: Preparation of N-[4-chloro-3-(trifluoromethyl)phenyl]-N′-{4-[2-carbamoyl-(4-pyridyloxy)]phenyl} urea

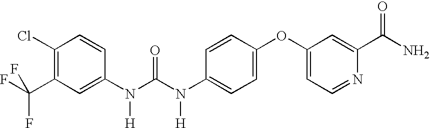

A mixture of 4-chloro-5-trifluoromethylaniline (451 mg, 2.31 mmol, 1.1 equiv.) and 1,1′-carbonyl diimidazole (419 mg, 2.54 mmol, 1.2 equiv.) in anh dichloroethane (5.5 mL) is stirred under argon at 65° C. for 16 h. Once cooled to room temperature, a solution of 4-(4-aminophenoxy)-2-pyridinecarboxamide (480 mg, 2.09 mmol) in anh THF (4.0 mL) is added, and the reaction mixture is stirred at 60° C. for 4 h. The reaction mixture is poured into EtOAc, and the organic layer is washed with water (2×) and a saturated NaCl solution (1×), dried (MgSO₄), filtered, and evaporated in vacuo. Purification using MPLC chromatography (Biotage®; gradient from 100% EtOAc to 2% MeOH/EtOAc) gave N-[4-chloro-3-(trifluoromethyl)phenyl]-N′-{4-[2-carbamoyl-(4-pyridyloxy)]phenyl} urea as a white solid (770 mg, 82%): TLC (EtOAc) R_{f} 0.11, 100% ethyl acetate ¹H-NMR (DMSO-d₆) δ 9.21 (s, 1H), 8.99 (s, 1H), 8.50 (d, J=5.6 Hz, 1H), 8.11 (s, 1H), 8.10 (s, 1H), 7.69 (broad s, 1H), 7.64 (dd, J=8.2 Hz, 2.1 Hz, 1H), 7.61 (s, 1H), 7.59 (d, J=8.8 Hz, 2H), 7.39 (d, J=2.5 Hz, 1H), 7.15 (d, J=8.9 Hz, 2H), 7.14 (m, 1H); MS LC-MS (MH⁺=451). Anal. calcd for C₂₀H₁₄ClF₃N₄O₃: C, 53.29% H, 3.13% N, 12.43%. Found: C, 53.33% H, 3.21% N, 12.60%.

Other methods of preparing N-[4-chloro-3-(trifluoromethyl)phenyl]-N′-{4-[2-carbamoyl-(4-pyridyloxy)]phenyl} urea are described in Bankston et al. “A Scaleable Synthesis of BAY 43-9006: A Potent Raf Kinase Inhibitor for the Treatment of Cancer” Org. Proc. Res. Dev. 2002, 6(6), 777-781, and WO 00/42012 and WO 00/41698.

An example of the preparation of sorafenib in the polymorph II is follows:

903 g of 4-{4-[({[4-chloro-3-(trifluoromethyl)phenyl]amino}carbonyl)amino]phenoxy}-N-methylpyridine-2-carboxamide, prepared as described above, are initially charged in 2700 ml of ethanol. 451.7 g of p-toluenesulfonic acid monohydrate are dissolved in 1340 g of ethanol and added dropwise at room temperature. The suspension is stirred at room temperature for 1 hour, then filtered off with suction, and the residue is washed three times with 830 ml each time of ethanol. The drying is effected at 50° C. under reduced pressure with supply of air. 1129.6 g of the title compound in the polymorph II are obtained.

An example of the preparation of sorafenib in the polymorph I is as follows:

Heating 5 mg of “Sorafenib,”[tosylate salt of 4-{5-[({[4-chloro-3-(trifluoromethyl)phenyl]amino}carbonyl)amino]-phenoxy}-N-methylpyridine-2-carboxamide] in the polymorph II to 200° C. at a heating rate of 20° C./min and subsequently cooling to room temperature at a cooling rate of 2° C./min. The sample is tested thermoanalytically (DSC) and corresponds to the title compound in the polymorph I.

The specific dose level and frequency of dosage may vary, depending upon a variety of factors, including the activity of the active agent, its metabolic stability and length of action, rate of excretion, mode and time of administration, the age, body weight, health condition, gender, diet, baseline hematologic and biologic parameters (e.g., WBCs, granulocytes, platelets, hemoglobin, creatinine, bilirubin, albumin, etc.), etc., of the subject, and the severity, intensity, stage of the cancer, primary site of cancer, size of cancer lesion, presence or extent of metastases, surgical status, disease progression (i.e., aggressive), etc. of the disease.

The compound of the present invention can be administered in any form by any effective route, including, e.g., oral, parenteral, enteral, intraperitoneal, topical, transdermal (e.g., using any standard patch), ophthalmic, nasally, local, non-oral, such as aerosal, spray, inhalation, subcutaneous, intravenous, intramuscular, buccal, sublingual, rectal, vaginal, intra-arterial, intrathecal, intratumoral, etc. Sorafenib can be administered directly to the site of a tumor, either pre- or post-operatively. It can be administered alone, or in combination with any ingredient(s), active or inactive.

Sorafenib can be administered by the oral route using the pharmaceutical composition of the present invention will generally range, based on body weight, from about 0.01 mg/kg to about 50 mg/kg; from about 1 mg/kg to about 40 mg/kg; from about 5 mg/kg to about 30 mg/kg; from about 10 to about 25 mg/kg; about 10 mg/kg; about 20 mg/kg; about 25 mg/kg; about 30 mg/kg; etc.

Any suitable dosing interval can be used in accordance with the present invention. For example, the compound can be administered once, twice (BID), three, four, etc., times a day. For example, about 100, about 200, about 400 mg, about 500 mg, about 600 mg, or about 800 mg can be administered one, twice, or three times daily.

Sorafenib can be administered at any suitable time. For example, it can be administered routinely as other chemotherapeutic agents; it can be administered as a bolus prior to a surgical intervention; prior to or after radiation, radiofrequency ablation and other energy treatments; post-operatively; pre-operatively; etc.

Sorafenib can be further combined with any other suitable additive or pharmaceutically acceptable carrier. Such additives include any of those used conventionally, such as those described in Remington: The Science and Practice of Pharmacy (Gennaro and Gennaro, eds, 20th edition, Lippincott Williams & Wilkins, 2000); Theory and Practice of Industrial Pharmacy (Lachman et al., eds., 3rd edition, Lippincott Williams & Wilkins, 1986); Encyclopedia of Pharmaceutical Technology (Swarbrick and Boylan, eds., 2nd edition, Marcel Dekker, 2002).

The compounds can be in any suitable form, without limitation. Forms suitable for oral use, include, but are not limited to, tablets, troches, lozenges, aqueous or oily suspensions, dispersible powders or granules, emulsions, hard or soft capsules, solutions, syrups and elixirs. Compositions intended for oral use may be prepared according to any method known to the art for the manufacture of pharmaceutical compositions.

Compounds can be formulated with other ingredients, e.g., “pharmaceutically acceptable carriers” or “excipients” to indicate they are combined with the active drug and can be administered safely to a subject for therapeutic purposes. These include, but are not limited to, antioxidants, preservatives, dyes, tablet-coating compositions, plasticizers, inert carriers, excipients, polymers, coating materials, osmotic barriers, devices and agents which slow or retard solubility, etc.

Compositions intended for oral use may be prepared according to any suitable method known to the art for the manufacture of pharmaceutical compositions. Such compositions may contain one or more agents selected from the group consisting of diluents, sweetening agents, flavoring agents, coloring agents and preserving agents in order to provide palatable preparations.

Non-toxic pharmaceutically acceptable excipients that are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; and binding agents, for example magnesium stearate, stearic acid or talc.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions containing the active materials in admixture with excipients suitable for the manufacture of aqueous suspensions may also be used. Such excipients are suspending agents, for example sodium carboxymethylcellulose, methylcellulose, hydroxypropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethylene oxycetanol, or condensation products of ethylene oxide with partial esters derived from fatty acids and hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, flavoring and coloring agents, may also be present.

The compounds may also be in the form of non-aqueous liquid formulations, e.g., oily suspensions which may be formulated by suspending the active ingredients in a vegetable oil, for example arachis oil, olive oil, sesame oil or peanut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The compounds may also be administered in the form of suppositories for rectal or vaginal administration of the drug. These compositions can be prepared by mixing the drug with a suitable non-irritating excipient which is solid at ordinary temperatures but liquid at the rectal temperature or vaginal temperature and will therefore melt in the rectum or vagina to release the drug. Such materials include cocoa butter and polyethylene glycols.

Compounds of the invention may also be administrated transdermally using methods known to those skilled in the art (see, for example: Chien; “Transdermal Controlled Systemic Medications”; Marcel Dekker, Inc.; 1987. Lipp et al. WO94/04157). For example, a solution or suspension of a compound of Formula I in a suitable volatile solvent optionally containing penetration enhancing agents can be combined with additional additives known to those skilled in the art, such as matrix materials and bacteriocides. After sterilization, the resulting mixture can be formulated following known procedures into dosage forms. In addition, on treatment with emulsifying agents and water, a solution or suspension of a compound of Formula I may be formulated into a lotion or salve.

Suitable solvents for processing transdermal delivery systems are known to those skilled in the art, and include lower alcohols such as ethanol or isopropyl alcohol, lower ketones such as acetone, lower carboxylic acid esters such as ethyl acetate, polar ethers such as tetrahydrofuran, lower hydrocarbons such as hexane, cyclohexane or benzene, or halogenated hydrocarbons such as dichloromethane, chloroform, trichlorotrifluoroethane, or trichlorofluoroethane. Suitable solvents may also include mixtures of one or more materials selected from lower alcohols, lower ketones, lower carboxylic acid esters, polar ethers, lower hydrocarbons, halogenated hydrocarbons.

Suitable penetration enhancing materials for transdermal delivery system are known to those skilled in the art, and include, for example, monohydroxy or polyhydroxy alcohols such as ethanol, propylene glycol or benzyl alcohol, saturated or unsaturated C8-C18 fatty alcohols such as lauryl alcohol or cetyl alcohol, saturated or unsaturated C8-C18 fatty acids such as stearic acid, saturated or unsaturated fatty esters with up to 24 carbons such as methyl, ethyl, propyl, isopropyl, n-butyl, sec-butyl, isobutyl, tertbutyl or monoglycerin esters of acetic acid, capronic acid, lauric acid, myristinic acid, stearic acid, or palmitic acid, or diesters of saturated or unsaturated dicarboxylic acids with a total of up to 24 carbons such as diisopropyl adipate, diisobutyl adipate, diisopropyl sebacate, diisopropyl maleate, or diisopropyl fumarate. Additional penetration enhancing materials include phosphatidyl derivatives such as lecithin or cephalin, terpenes, amides, ketones, ureas and their derivatives, and ethers such as dimethyl isosorbid and diethyleneglycol monoethyl ether. Suitable penetration enhancing formulations may also include mixtures of one or more materials selected from monohydroxy or polyhydroxy alcohols, saturated or unsaturated C8-C18 fatty alcohols, saturated or unsaturated C8-C18 fatty acids, saturated or unsaturated fatty esters with up to 24 carbons, diesters of saturated or unsaturated discarboxylic acids with a total of up to 24 carbons, phosphatidyl derivatives, terpenes, amides, ketones, ureas and their derivatives, and ethers.

Suitable binding materials for transdermal delivery systems are known to those skilled in the art and include polyacrylates, silicones, polyurethanes, block polymers, styrenebutadiene copolymers, and natural and synthetic rubbers. Cellulose ethers, derivatized polyethylenes, and silicates may also be used as matrix components. Additional additives, such as viscous resins or oils may be added to increase the viscosity of the matrix.

Compositions comprising precursors can also be formulated for controlled release, where release of the active ingredient is regulated or modulated to achieve a desired rate of delivery into the systemic circulation. A controlled release formulation can be pulsed, delayed, extended, slow, steady, immediate, rapid, fast, etc. It can comprise one or more release formulations, e.g. extended- and immediate-release components. Extended delivery systems can be utilized to achieve a dosing internal of once every 24 hours, once every 12 hours, once every 8 hours, once every 6 hours, etc. The dosage form/delivery system can be a tablet or a capsule suited for extended release, but a sustained release liquid or suspension can also be used. A controlled release pharmaceutical formulation can be produced which maintains the release of, and or peak blood plasma levels of sorafenib.

In preferred solid oral pharmaceutical compositions according to the invention, at least 80% of the sorafenib exists in the stable polymorph I form and most preferably sorafenib is present in a micronized form.

Micronization can be achieved by standard milling methods, preferably by air chat milling, known to a skilled person. The micronized form can have a mean particle size of from 0.5 to 10 μm, preferably from 1 to 6 μm, more preferably from 1 to 3 μm. The indicated particle size is the mean of the particle size distribution measured by laser diffraction known to a skilled person (measuring device: HELOS, Sympatec).

Pharmaceutical compositions which are preferred comprise sorafenib, a compound of formula (I) or another pharmaceutically acceptable salt of a compound of formula I in a portion of at least 40%, preferably at least 45%, more preferably at least 50%, even more preferably at least 55%, by weight of the composition. Amounts of at least 62%, or at least 69%, or at least 75% by weight of the composition can be used under certain circumstances. Methods for preparing such formulations are disclosed in provisional application Ser. No. 60/658,827, filed Mar. 7, 2005, which is incorporated herein by reference.

Without further elaboration, it is believed that one skilled in the art, using the preceding description, can utilize the present invention to its fullest extent. The following preferred specific embodiments are, therefore, to be construed as merely illustrative, and not limitative of the remainder of the disclosure in any way whatsoever. The entire disclosure of all applications, patents and publications, cited above and in the figures are hereby incorporated by reference in their entirety.

### EXAMPLES

### Generation of c-KIT-Expressing Ba/F3 Cell Lines

The cDNA encoding full length human c-KIT with a deletion in exon 11 that removed amino acid residues 557-558 was ligated into the mammalian expression vector pClneo (Promega). Gleevec-resistant variants of the KIT exon 11 deletion mutants were generated using conventional methods. All mutations were confirmed by DNA sequencing.

The expression vectors encoding the c-KIT exon 11 mutant or its Gleevec-resistant mutant variants were transfected into Ba/F3 cells by electroporation using an optimized protocol developed by Amaxa Biosystems. Selective pressure was applied to the transfected cells by removing IL-3 from the culture medium. After IL-independent populations, further selective pressure was applied by also growing the cells in the presence of 1 mg/mL G418. The resulting stable pools of Ba/F3 cells were found to express c-KIT by western blot using an antibody specific for c-KIT. The stable pools were further characterized by sequencing genomic DNA to confirm the presence of the transfected c-KIT cDNA.

Cell Proliferation Assay

This assay utilizes cellular ATP as a marker for cell proliferation/viability. On day 1, Ba/F3 cells were plated in 96 well dishes (Costar 3603) at 10,000 cells per well in 10% FBS in RPMI medium with 1 mg/ml G418. Test compounds, serially diluted in the same medium at 10× for an eight-point dose response to give rise to final concentrations ranging from 0.6 to 10,000 nM, were added to the cells. Plates were then incubated in a 5% CO₂ incubator at 37° C. for 3 days. After 72 h, 100 microliters of lysis/luciferase reagent (CellTiter-Glo, Promega G7573) was added to each well. The cells were then incubated on a shaker for 5 minutes at room temperature, and luminescence was measured on a Victor 5 (Perkin Elmer) spectrophotometer. Growth inhibition was measured by comparing luminescence signal from treated vs. untreated cells in assay plates, and the IC50 analysis of cell proliferation inhibition by compounds was analyzed using Analyze 5 in-house software. IC50 values obtained for Gleevec and Nexavar (sorafenib) sorafenib (BAY 43-9006) in the various c-KIT-expressing Ba/F3cell lines are summarized in Table 2. The IC50 values are mean values calculated from at least three experiments.

TABLE 1
K P M Y E V Q W K V V E E I N G N N
50 51 52 53 54 55 56 57 58 59 60 61 62 63 64 65 66
Y V Y I D P T Q L P Y D H
67 68 69 70 71 72 73 74 75 76 77 78 79 80

TABLE 2

| | Exon 11 | Exon 11/Gleevec-resistant Double Mutant | | | | |
|---|---|---|---|---|---|---|
| Inhibitor | (WT) | V654A | T670I | D816G | N822K | Y823D |
| Gleevec | 5 | 168 | >10,000 | 87 | 221 | 295 |
| BAY 43-9006 | 3 | 46 | 13 | 8 | 17 | 36 |

1. A method of treating a cancer in a subject in need thereof, wherein said cancer was initially sensitive to KIT tyrosine kinase inhibitor and acquired resistance to said KIT tyrosine kinase inhibitor, said method comprising:
administering to said subject, an effective amount of the tosylate salt of the compound N-[4-chloro-3-(trifluoromethyl)phenyl]-N′-{4-[2-carbamoyl-1-oxo-(4-pyridyloxy)]phenyl} urea of the formula I below including all polymorphs, hydrates, solvates or combinations thereof.

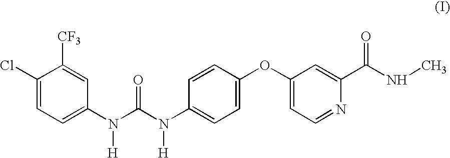

2. A method as in claim 1 wherein the cancer has acquired resistance to one of the following KIT inhibitors:
imatinib mesylate, derivatives of imatinib mesylate, salts of imatinib mesylate; PP1(4-Amino-5-(4-methylphenyl)-7-(t-butyl)pyrazolo[3,4-d]pyrimidine); MLN518 (CT53518); PD180970; SU11248I SU5416; SU5414; SU6597; SU6663 or SU6561.

3. A method as in claim 1 wherein said cancer is one or more of a malignant gastrointestinal stromal tumor (GIST), a benign gastrointestinal stromal tumor (GIST), a mesenchymal tumor of the intestinal tract, chronic myelogenous leukemia (CML), a mast cell tumor, SCLC, a germ cell tumors, breast cancer, and/or neuroblastoma.

4. A method as in claim 1 wherein the cancer has acquired resistance to imatinib mesylate.

5. A method of claim 1, wherein said acquired resistance of said cancer is associated with a secondary mutation in a KIT gene mutated in the primary tumor.

6. A method of claim 5, wherein said secondary mutation is in the kinase catalytic domain.

7. A method as in claim 5 wherein the mutation is in Exons 13, 14, and or 17.

8. A method as in claim 5 wherein the mutation is at residues 654, 670, 716.816, 820, 822, and 823.

9. A method as in claim 5 wherein the mutation is at residues 650-654.

10. A method as in claim 5 wherein the mutation is at residues 670-674.

11. A method as in claim 5 wherein the mutation is at residues 816-824.

12. A method as in claim 5 wherein the secondary mutation is one or more of V654A (Exon 13), T670I (Exon 14), T670E, D716N, S709F (Exon 14), D816G, D816E (Exon 17), D820E, D820Y, D820G N822K, Y823D (Exon 17), or deletions and other amino acid substitutions at such positions or adjacent positions.

13. A method as in claim 5 wherein the secondary mutation is one or more of
i) deletion of amino acid residues 557-558;
ii) deletion of amino acid residues 551-555;
iii) deletion of amino acid residues 550-558;
iv) deletion of amino acid residues 559-560;
v) deletion of amino acid residues 557-561;
vi) deletion of amino acid residues 554-558;
vii) deletion of amino acid residues 552-557;
viii) mutations at residue 559, including V559D, V559A, or V559G;
ix) mutations at residue 560, including V560D, V560E, or V560G;
x) W557S, alone, or in combination with a deletion of amino acids 552-556;
xi) mutations at amino acid residue 557, including W557R; and
xii) mutations at amino acid residue 576, including L576P.

14. A method as in claim 5 wherein the secondary mutation is deletion of residues 557-558 and at least one of the following mutations: V654A, T670I, D820Y, N822K, or Y823D.

15. A method of treating a cancer in a subject in need thereof said cancer having a primary and/or secondary KIT gene mutation in the primary tumor, said method comprising:
administering to said subject, an effective amount of the tosylate salt of the compound N-[4-chloro-3-(trifluoromethyl)phenyl]-N′-{4-[2-carbamoyl-1-oxo-(4-pyridyloxy)]phenyl} urea of the formula I below including all polymorphs, hydrates, solvates or combinations thereof.

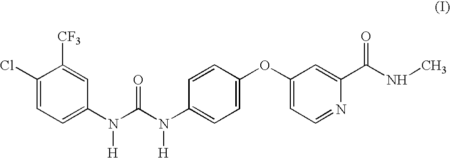

16. A method of claim 15, wherein said primary and/or secondary KIT gene mutation in the primary tumor is associated with acquired resistance of said cancer to KIT tyrosine kinase inhibitors.

17. A method of claim 15, wherein said secondary mutation is in the kinase catalytic domain.

18. A method as in claim 15 wherein the mutation is in Exons 13, 14, and or 17.

19. A method as in claim 15 wherein the mutation is at residues 654, 670, 716, 816, 820, 822, and 823.

20. A method as in claim 15 wherein the mutation is at residues 650-654.

21. A method as in claim 15 wherein the mutation is at residues 670-674.

22. A method as in claim 15 wherein the mutation is at residues 816-824.

23. A method as in claim 15 wherein the secondary mutation is one or more of V654A (Exon 13), T670I (Exon 14), T670E, D716N, S709F (Exon 14), D816G, D816E (Exon 17), D820E, D820Y, D820G N822K, Y823D (Exon 17), or deletions and other amino acid substitutions at such positions or adjacent positions.

24. A method as in claim 15 wherein the secondary mutation is one or more of
i) deletion of amino acid residues 557-558;
ii) deletion of amino acid residues 551-555;
iii) deletion of amino acid residues 550-558;
iv) deletion of amino acid residues 559-560;
v) deletion of amino acid residues 557-561;
vi) deletion of amino acid residues 554-558;
vii) deletion of amino acid residues 552-557;
viii) mutations at residue 559, including V559D, V559A, or V559G;
ix) mutations at residue 560, including V560D, V560E, or V560G;
x) W557S, alone, or in combination with a deletion of amino acids 552-556;
xi) mutations at amino acid residue 557, including W557R; and
xii) mutations at amino acid residue 576, including L576P.

25. A method as in claim 15 wherein the secondary mutation is deletion of residues 557-558 and at least one of the following mutations: V654A, T670I, D820Y, N822K, or Y823D.

26. A method of treating a cancer in a subject in need thereof said cancer having a primary and/or secondary KIT gene mutation associated with resistance or acquired resistance to imatinib mesylate, derivatives of imatinib mesylate or salts of imatinib mesylate, said method comprising:
administering to said subject, an effective amount of the tosylate salt of the compound N-[4-chloro-3-(trifluoromethyl)phenyl]-N′-{4-[2-carbamoyl-1-oxo-(4-pyridyloxy)]phenyl} urea of the formula I below including all polymorphs, hydrates, solvates or combinations thereof.

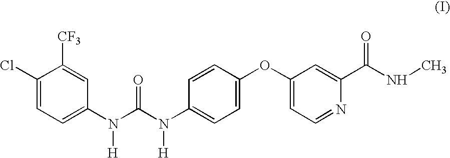

27. A method for treating cancer in a human subject with imatinib mesylate, derivatives of imatinib mesylate, or salts of imatinib mesylate, which additionally comprises:
administering to said human subject, an effective amount of the tosylate salt of the compound N-[4-chloro-3-(trifluoromethyl)phenyl]-N′-{4-[2-carbamoyl-1-oxo-(4-pyridyloxy)]phenyl} urea of the formula I below including all polymorphs, hydrates, solvates or combinations thereof.

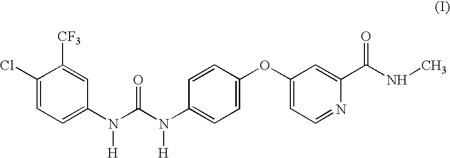

28. A method as in claim 1 wherein the cancer which is treated is:
Accelerated Phase Chronic Myelogenous Leukemia; Acute Erythroid Leukemia; Acute Lymphoblastic Leukemia; Acute Lymphoblastic Leukemia in Remission; Acute Lymphocytic Leukemia; Acute Monoblastic and Acute; Monocytic Leukemia; Acute Myelogenous Leukemia; Acute Myeloid Leukemia; Adenocarcinoma of the Prostate; Adenoid Cystic Carcinoma of the Head and Neck; Advanced Gastrointestinal Stromal Tumor; Agnogenic Myeloid; Metaplasia; Anaplastic Oligodendroglioma; Astrocytoma; B-Cell Adult Acute Lymphoblastic Leukemia; Blastic Phase Chronic Myelogenous Leukemia; Bone Metastases; Brain Tumor; Breast Cancer; Cancer; Central Nervous System Cancer; Childhood Acute Lymphoblastic Leukemia; Childhood Acute Lymphoblastic Leukemia in Remission; Childhood Central Nervous System Germ Cell Tumor; Childhood Chronic Myelogenous Leukemia; Childhood Soft Tissue Sarcoma; Chordoma; Chronic Eosinophilic Leukemia (CEL); Chronic Idiopathic Myelofibrosis; Chronic Myelogenous Leukemia; Chronic Myeloid Leukemia; Chronic Myelomonocytic Leukemia; Chronic Phase Chronic Myelogenous Leukemia; Colon Cancer; Colorectal Cancer; Dermatofibrosarcoma; Dermatofibrosarcoma Protuberans (DFSP); Desmoid Tumor; Eosinophilia; Epidemic Kaposi's Sarcoma; Essential Thrombocythemia; Ewing's Family of Tumors; Extensive Stage Small Cell Lung Cancer; Fallopian Tube Cancer; Familiar Hypereosinophilia; Fibrosarcoma; Gastric Adenocarcinoma; Gastrointestinal Neoplasm; Gastrointestinal Stromal Tumor; Glioblastoma; Glioma; Gliosarcoma; Grade I Meningioma; Grade II Meningioma; Grade III Meningioma; Hematopoietic and Lymphoid Cancer; High-Grade Childhood Cerebral Astrocytoma; Hypereosinophilic Syndrome; Idiopathic Pulmonary Fibrosis; L1 Adult Acute Lymphoblastic Leukemia; L2 Adult Acute Lymphoblastic Leukemia; Leukemia, Lymphocytic, Acute L2; Leukemia, Myeloid, Chronic; Leukemia, Myeloid, Chronic Phase; Liver Dysfunction and Neoplasm; Lung Disease; Lymphoid Blastic Phase of Chronic Myeloid Leukemia; Male Breast Cancer; Malignant Fibrous Histiocytoma; Mastocytosis; Meningeal Hemangiopericytoma; Meningioma; Meningioma; Meningioma; Metastatic Cancer; Metastatic Solid Tumors; Myelofibrosis; Myeloid Leukemia, Chronic; Myeloid Leukemia, Chronic Accelerated-Phase; Myeloid Leukemia, Chronic, Chronic-Phase; Myeloid Metaplasia; Myeloproliferative Disorder (MPD) with Eosinophilia; Neuroblastoma; Non-T, Non-B Childhood Acute Lymphoblastic Leukemia; Oligodendroglioma; Osteosarcoma; Ovarian Germ Cell Tumor; Ovarian Low Malignant Potential Tumor; Ovarian Neoplasms; Pancreatic Cancer; Pelvic Neoplasms; Peritoneal Cavity Cancer; Peritoneal Neoplasms; Philadelphia Chromosome Positive Chronic Myelogenous Leukemia; Philadelphia Positive Acute Lymphoblastic Leukemia; Philadelphia Positive Chronic Myeloid Leukemia in Myeloid Blast Crisis; Polycythemia Vera; Pulmonary Fibrosis; Recurrent Adult Brain Tumor; Recurrent Adult Soft Tissue Sarcoma; Recurrent Breast Cancer; Recurrent Colon Cancer; Recurrent Esophageal Cancer; Recurrent Gastric Cancer; Recurrent Glioblastoma Multiforme (GBM); Recurrent Kaposi's Sarcoma; Recurrent Melanoma; Recurrent Merkel Cell Carcinoma; Recurrent Ovarian Epithelial Cancer; Recurrent Pancreatic Cancer; Recurrent Prostate Cancer; Recurrent Rectal Cancer; Recurrent Salivary Gland Cancer; Recurrent Small Cell Lung Cancer; Recurrent Tumors of the Ewing's Family; Recurrent Uterine Sarcoma; Relapsing Chronic Myelogenous Leukemia; Rheumatoid Arthritis; Salivary Gland Adenoid Cystic Carcinoma; Sarcoma; Small Cell Lung Cancer; Stage II Melanoma; Stage II Merkel Cell Carcinoma; Stage III Adult Soft Tissue Sarcoma; Stage III Esophageal Cancer; Stage III Merkel Cell Carcinoma; Stage III Ovarian Epithelial Cancer; Stage III Pancreatic Cancer; Stage III Salivary Gland Cancer; Stage IIIB Breast Cancer; Stage IIIC Breast Cancer; Stage IV Adult Soft Tissue Sarcoma; Stage IV Breast Cancer; Stage IV Colon Cancer; Stage IV Esophageal Cancer; Stage IV Gastric Cancer; Stage IV Melanoma; Stage IV Ovarian Epithelial Cancer; Stage IV Prostate Cancer; Stage IV Rectal Cancer; Stage IV Salivary Gland Cancer; Stage IVA Pancreatic Cancer; Stage IVB Pancreatic Cancer; Systemic Mastocytosis; T-Cell Childhood Acute Lymphoblastic Leukemia; Testicular Cancer; Thyroid Cancer; Unresectable or Metastatic Malignant Gastrointestinal Stromal Tumor (GIST); Unspecified Adult Solid Tumor; Untreated Childhood Brain Stem Glioma; Uterine Carcinosarcoma, and Uterine Sarcoma.

29. A method of treating a cancer in a subject who has acquired resistance to imatinib, comprising:
administering an effective amount of sorafenib to said subject.
